(19) United States
(12) Patent Application Publication
(10) Pub. No.: US 2006/0173205 A1
(43) Pub. Date: Aug. 3, 2006

(54) Process for producing w-cyanoaldehyde compound

(76) Inventors: Yasuhisa Fukuda, Yamaguchi (JP); Kenji Hirotsu, Yamaguchi (JP); Tadashi Murakami, Yamaguchi (JP); Noboru Kakeya, Yamaguchi (JP); Takashi Honma, Yamaguchi (JP)

Correspondence Address: BIRCH STEWART KOLASCH & BIRCH, PO BOX 747, FALLS CHURCH, VA, 22040-0747, US

(21) Appl. No.: 10/548,800

(22) PCT Filed: Mar. 19, 2004
(86) PCT No.: PCT/JP04/03758

§ 371 Date: Sep. 12, 2005

(30) Foreign Application Priority Data
Mar. 19, 2003 (JP) 2003-075960

(51) Int. Cl. C07C 253/00

(52) U.S. Cl. 558/308

(57) ABSTRACT

The present invention is to provide a process for preparing an ω-cyanoaldehyde compound which comprises bringing a 2-alkoxycycloalkanone oxime compound into contact with a solid acid(s).

### TECHNICAL FIELD

The present invention relates to a novel process for preparing an ω-cyanoaldehyde compound. The ω-cyanoaldehyde compound is a useful compound as a starting material for various diamines, aminonitriles, and the like. For example, 12-oxo-4,8-dodecadienenitrile can be led to dodecamethylenediamine which is useful as a starting material of 1212 Nylon, etc. by a reductive amination.

### BACKGROUND ART

As prior art techniques which relate to the preparation process of the present invention, there has been disclosed a process for preparing an ω-cyanoaldehyde compound by reacting an α-alkoxy oxime compound with a halogen and an organic phosphorus compound such as Ph₃P, etc. (for example, see Japanese Patent Publication No. Sho. 43-015962).

In addition, there have been disclosed a method for preparing an ω-cyanoaldehyde compound by reacting an α-alkoxy oxime compound with phosphorus pentachloride (for example, see J. Am. Chem. Soc. (1966), 88, p. 3168), a method for preparing the same by reacting a 2-methoxy-5,9-cyclododecadienone oxime with phosphorus pentachloride (for example, see J. Org. Chem. USSR (1980), 16, p. 1534 and Zh. Org. Khim. (1980), 16 (9), p. 1813), and a method for preparing a 7-cyanoheptanal by reacting a 2-methoxycyclooctenone oxime with phosphorus pentachloride (for example, Org. Syn. (1969), 49, p. 27).

However, in these methods, a halogen or a phosphorus compound which is unstable and has potent toxicity is used, so that they cause corrosion of an apparatus, etc., and severe and strict attention for their handling are required.

Also, there has been disclosed a process for preparing an α-cyanoaldehyde compound by reacting 2-hydroxycyclohexanone oxime, 2-methoxy-5,9-cyclododecadienone oxime, etc. with formic acid or a carboxylic acid anhydride (for example, see Japanese Unexamined Patent Publications No. Hei. 09-040629, No. Hei. 09-003028, No. Hei. 14-088040), but the yield of the objective material is about 70 mol % so that it is not satisfied. Also, there is a problem that a complicated step such as distillation and purification to separate and recover formic acid, etc., utilized for the reaction.

The present invention is to solve the above-mentioned problems, and to provide a novel process for preparing an ω-cyanoaldehyde compound which is improved in operatability in the reaction, easy in recovering operation of an acid to be used as a catalyst or reuse of the same, and safety, and further, the objective compound can be obtained with high selectivity.

### DISCLOSURE OF THE INVENTION

The present invention relates to a process for preparing an ω-cyanoaldehyde compound which comprises bringing a 2-alkoxycycloalkanone oxime compound into contact with a solid acid(s).

### BEST MODE FOR CARRYING OUT THE INVENTION

In the following, the present invention is explained in detail.

The 2-alkoxycycloalkanone oxime compound which is a starting compound of the present invention can be prepared by reacting a corresponding 2-halogenocycloalkanone oxime compound and an alcohol, and a process for preparing 2-alkoxycyclododecadienone oxime is disclosed in Japanese Patent Publication No. Sho. 45-19902.

As the 2-alkoxycycloalkanone oxime compound, a 2-alkoxycycloalkanone oxime compound comprising a saturated or unsaturated cyclic hydrocarbon and having a carbon number of 6 to 12 is preferred, and a 2-alkoxycyclododecanone oxime compound having a carbon number of 12 is particularly preferred.

Incidentally, in the case of the 2-alkoxycycloalkanone oxime compound having at least one double bond, any of a cis-isomer or a trans-isomer, etc., may be used. These isomers may be used in admixture without any problem.

Also, the 2-alkoxycycloalkanone oxime compound may be a commercially available product or a synthesized product and used as such, or a purified product by crystallization, etc. can be used without any problem.

The alkoxy group in the 2-alkoxycycloalkanone oxime compound is not specifically limited, and preferably an alkoxy group having a carbon number of 1 to 7, particularly preferably a methoxy group and a butoxy group.

Specific compounds may be mentioned 2-methoxycyclopentanone oxime, 2-methoxycyclohexanone oxime, 2-methoxycyclohexenone oxime, 2-methoxycycloheptanone oxime, 2-methoxycyclooctanone oxime, 2-methoxycyclooctenone oxime, 2-methoxycyclononanone oxime, 2-methoxycyclodecanone oxime, 2-methoxycycloundecanone oxime, 2-methoxycyclododecanone oxime, 2-methoxycyclododecadienone oxime, 2-butoxycyclododecadienone oxime and the like. It is preferably 2-alkoxycyclododecadienone oxime compound, and particularly preferably 2-alkoxy-5,9-cyclododecadienone oxime. These compounds can be used alone or in combination of two or more kinds in admixture.

The solid acid(s) to be used in the present invention shows characteristics of a Brønsted acid or a Lewis acid while it is a solid state, and it is not specifically limited, and there may be mentioned zeolites such as β type zeolite (H-β zeolite, etc.), Y type zeolite (H-USY zeolite, etc.), mordenite, titanosilicate and MCM-22, etc. and a modified product thereof, oxides such as aluminum oxide and zinc oxide, etc., composite oxides such as SiO₂—Al₂O₃, SiO₂—TiO₂, etc., clay minerals such as kaolin, bentonite, activated clay, etc., ion exchange resins such as Amberlyst (Amberlyst®, available from Rhom & Haas AG; a sulfonic acid group is introduced into a styrene-divinylbenzene copolymer), Nafion (Nafion®, registered trademark by DuPont, strongly acidic ion exchange resin which is a copolymer of perfluorosulfonic acid and tetrafluoroethylene), etc. and a molded product in which these resins are carried on silica gel, etc., phosphates such as calcium phosphate, etc., sulfates such as sulfated zirconia, copper sulfate, etc., heteropoly acids, and the like. These solid acids may be used with one kind, or may be used in combination of two or more kinds.

They are preferably activated clay, Nafion® SAC-13, H-β zeolite and H-USY zeolite.

The present reaction is a novel reaction which forms an ω-cyanoaldehyde compound according to the reaction scheme shown in the following formula.

The method of bringing the solid acid(s) and the 2-alkoxycycloalkanone oxime compound into contact is not specifically limited, and there may be mentioned a gas-phase heterogeneous systems or a heterogeneous system in a liquid phase.

An amount of the solid acid(s) to be used is preferably 0.01% by weight or more based on the amount of the 2-alkoxycyclododecanone oxime compound, more preferably 1 to 300% by weight, further preferably 10 to 200% by weight.

In a liquid phase heterogeneous system, the used solid acid(s) can be easily separated from the reaction system after completion of the reaction. Also, it is possible that the solid acid(s) can be used until it is deactivated or the deactivated solid acid(s) can be regenerated by a heat treatment, etc. 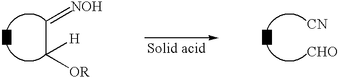
wherein R represents an alkyl group having 1 to 7 carbon atoms, Y represents a saturated or unsaturated alkylene group having 4 to 10 carbon atoms.

Moreover, the reaction conditions of heterogeneous systems in a liquid phase are, in general, preferably selected to carry out to bring the 2-alkoxycycloalkanone oxime compound into contact with the solid acid(s) in the presence of an organic solvent. The solvent is not specifically limited so long as it is a solvent inactive to the present reaction, and there may be mentioned aliphatic alcohols such as methanol, ethanol, etc., nitrites such as acetonitrile, benzonitrile, etc., aliphatic halogenated hydrocarbons such as methylene chloride, carbon tetrachloride, etc., ethers such as diethyl ether, dioxane, etc., aliphatic hydrocarbons such as hexane, heptane, etc., aromatic hydrocarbons such as toluene, chlorobenzene, nitrobenzene, etc., ketones such as acetone, methyl ethyl ketone, methyl isopropyl ketone, methyl isobutyl ketone, diethyl ketone, diisopropyl ketone, cyclohexanone, etc., aliphatic carboxylic acids such as acetic acid, propionic acid, etc., sulfolane, dimethylsulfoxide, etc. It is preferably ketones and nitrites, more preferably ketones. Among the ketones, a ketone compound having a methyl group, acetone, methyl ethyl ketone, methyl isopropyl ketone, methyl isobutyl ketone, etc., are preferred.

These solvents can be generally used in an amount of 0 to 100-fold weight, preferably 1 to 50-fold weight based on the amount of the 2-alkoxycycloalkanone oxime compound.

The reaction temperature is not specifically limited so long as the reaction is carried out at a boiling point of the solvent to be used or lower, and it can be carried out preferably at 40 to 200° C., more preferably at 50 to 170° C.

Also, the reaction pressure is usually a normal pressure, but the reaction may be carried out with slightly pressurized.

The reaction device is also not specifically limited, and the reaction can be carried out in a reactor equipped with a usual stirring device, and the like.

The reaction time may vary depending on the above-mentioned amount of the solid acid(s) to be used, the reaction conditions such as a reaction temperature, etc., but it can be usually carried out for 0.01 to 24 hours.

The reaction mixture containing the ω-cyanoaldehyde compound obtained in the present invention is subjected to removal of the solid acid(s), by a simple and easy operation such as filtration, etc., and then, the reaction mixture is subjected to separation and purification operation such as distillation, crystallization, column chromatography, etc. so that the ω-cyanoaldehyde compound can be isolated.

### EXAMPLE

Next, the present invention is explained more specifically by referring to Examples.

### Example 1

In 10 ml of methyl isobutyl ketone was dissolved 0.41 g (1.84 mmol) of 2-methoxy-5,9-cyclododecadienone oxime, 0.41 g of Nafion® SAC-13 was added to the solution, and the resulting mixture was reacted under reflux (105 to 107° C.), for 45 minutes. When the reaction mixture was quantitated by using GC, a conversion rate of the 2-methoxy-5,9-cyclododecadienone oxime was 100 mol %, and 12-oxo-4,8-dodecadienenitrile was found to be formed with a selectivity of 85 mol %. The results are shown in Table 1.

### Examples 2 to 8

Reaction was carried out in accordance with Example 1 except for changing a kind and an amount of the solvent, and various conditions such as a reaction temperature, etc. as shown in Table 1. The results are also shown in Table 1.

TABLE 1

| | Starting material | | | | | | |
|---|---|---|---|---|---|---|---|
| | 2-alkoxy-5,9- | | | | | | |
| | cyclododeca- | | | Reaction | | Oxime | CNCHO^{″} |
| | dienone oxime | Solid acid (% by | | temperature | Reaction | conversion | Selectivity |
| | (mmol) | weight) | Solvent (ml) | (° C.) | time (min) | rate (mol %) | (mol %) |
| Example | 2-methoxy- | Nafion ®SAC-13 | Methyl isobutyl | Reflux | 45 | 100 | 85 |
| 1 | (1.84) | 100% by weight | ketone (10) | (105-107° C.) | | | |
| Example | 2-methoxy- | Nafion ®SAC-13 | Benzonitrile (10) | 128-132° C. | 240 | 100 | 57 |
| 2 | (1.70) | 100% by weight | | | | | |
| Example | 2-methoxy- | Nafion ®SAC-13 | Benzonitrile (5) | 103-107° C. | 120 | 76 | 55 |
| 3 | (0.94) | 100% by weight | | | | | |
| Example | 2-methoxy- | Nafion ®SAC-13 | Xylene (10) | 125° C. | 240 | 72 | 49 |
| 4 | (1.84) | 100% by weight | | | | | |
| Example | 2-methoxy- | Nafion ®SAC-13 | Toluene (12) | 105° C. | 480 | 76 | 46 |
| 5 | (1.84) | 100% by weight | | | | | |
| Example | 2-methoxy- | Nafion ®SAC-13 | Sulfolane (5) | 105-107° C. | 120 | 69 | 55 |
| 6 | (0.90) | 100% by weight | | | | | |
| Example | 2-methoxy- | Nafion ®SAC-13 | Benzonitrile (10) | Reflux | 45 | 100 | 64 |
| 7 | (1.84) | 100% by weight | | (165° C.) | | | |
| Example | 2-methoxy- | Nafion ®SAC-13 | Methyl isobutyl | 65-67° C. | 240 | 93 | 75 |
| 8 | (0.90) | 100% by weight | ketone (5) | | | | |

An amount of a solid acid used (% by weight) is an amount based on the amount of the starting 2-alkoxy-5,9-cyclododecadienone oxime used.
CNCHO^{″}: 12-oxo-4,8-dodecadienenitrile

### Example 9

In 10 ml of methyl ethyl ketone was dissolved 0.40 g (1.79 mmol) of 2-methoxy-5,9-cyclododecadienone oxime, and 0.40 g of activated clay (F-24 available from Engelhard Corporation) and 0.40 g of diphenyl ether which is an internal standard substance were added to the mixture, and the resulting mixture was reacted under reflux (75 to 77° C.) for 2 hours. When the reaction mixture was quantitated by GC, a conversion rate of 2-methoxy-5,9-cyclododecadienone oxime was 100 mol %, and 12-oxo-4,8-dodecadienenitrile was quantitatively formed.

The results are also shown in Table 2 together.

### Examples 10 to 31

Reaction was carried out in accordance with Example 9 except for changing a kind and an amount of the solvent, and various conditions such as a reaction temperature, etc. as shown in Table 2. The results are also shown in Table 2 together.

TABLE 2

| | Starting material | | | | | Oxime | |
|---|---|---|---|---|---|---|---|
| | 2-Alkoxy-5,9-cyclo- | | | Reaction | | conversion | CNCHO^{″} |
| | dodecadienone | Solid acid | Solvent | temperature | Reaction | rate | selectivity |
| | oxime (mmol) | (% by weight) | (ml) | (° C.) | time (min) | (mol %) | (mol %) |
| Example | 2-methoxy- | activated clay | Methyl ethyl | Reflux | 120 | 100 | 100 |
| 9 | (1.79) | 100% by weight | ketone (10) | (75-77° C.) | | | |
| Example | 2-methoxy- | activated clay | Chlorobenzene (5) | 80-82° C. | 180 | 51 | 29 |
| 10 | (1.75) | 100% by weight | | | | | |
| Example | 2-methoxy- | activated clay | Anisole (5) | 80° C. | 180 | 62 | 23 |
| 11 | (1.75) | 100% by weight | | | | | |
| Example | 2-methoxy- | activated clay | Dichloroethane (5) | 80° C. | 180 | 45 | 29 |
| 12 | (0.94) | 95% by weight | | | | | |
| Example | 2-methoxy- | activated clay | Methyl isobutyl | Reflux | 30 | 99 | 81 |
| 13 | (1.79) | 100% by weight | ketone (5) | (105-107° C.) | | | |
| Example | 2-methoxy- | activated clay | Methyl isobutyl | Reflux | 420 | 54 | 72 |
| 14 | (1.79) | 25% by weight | ketone (10) | (105-107° C.) | | | |
| Example | 2-methoxy- | activated clay | Methyl isobutyl | Reflux | 240 | 99 | 80 |
| 15 | (1.79) | 50% by weight | ketone (10) | (105-107° C.) | | | |
| Example | 2-methoxy- | activated clay | Methyl isobutyl | Reflux | 30 | 95 | 89 |
| 16 | (1.79) | 100% by weight | ketone (10) | (105-107° C.) | | | |
| Example | 2-methoxy- | activated clay | Methyl isobutyl | Reflux | 10 | 96 | 94 |
| 17 | (1.88) | 260% by weight | ketone (10) | (105-107° C.) | | | |
| Example | 2-methoxy- | activated clay | Methyl isobutyl | Reflux | 120 | 100 | 86 |
| 18 | (1.79) | 100% by weight | ketone (20) | (105-107° C.) | | | |
| Example | 2-methoxy- | activated clay | Methyl isobutyl | 75-77° C. | 60 | 83 | 83 |
| 19 | (1.79) | 100% by weight | ketone (5) | | | | |
| Example | 2-methoxy- | activated clay | Diisopropyl | (105-107° C.) | 30 | 55 | 58 |
| 20 | (1.79) | 100% by weight | ketone (5) | | | | |
| Example | 2-methoxy- | activated clay | Cyclohexa- | (105-107° C.) | 30 | 97 | 71 |
| 21 | (1.79) | 100% by weight | none (5) | | | | |
| Example | 2-methoxy- | activated clay | Methyl isopro- | 79-80° C. | 120 | 98 | 92 |
| 22 | (1.79) | 100% by weight | pyl ketone (5) | | | | |
| Example | 2-methoxy- | activated clay | Acetone (5) | 50-55° C. | 300 | 51 | 92 |
| 23 | (1.79) | 100% by weight | | | | | |
| Example | 2-methoxy- | activated clay | Methyl ethyl | Reflux | 180 | 95 | 95 |
| 24 | (1.79) | 10% by weight | ketone (5) | (75-77° C.) | | | |
| Example | 2-methoxy- | activated clay | Methyl ethyl | Reflux | 120 | 100 | 96 |
| 25 | (1.79) | 25% by weight | ketone (5) | (75-77° C.) | | | |
| Example | 2-methoxy- | activated clay | Methyl ethyl | Reflux | 60 | 97 | 86 |
| 26 | (1.79) | 100% by weight | ketone (2) | (75-77° C.) | | | |
| Example | 2-methoxy- | activated clay | Methyl ethyl | Reflux | 60 | 99 | 96 |
| 27 | (1.79) | 100% by weight | ketone (5) | (75-77° C.) | | | |
| Example | 2-methoxy- | activated clay | Methyl ethyl | (62-67° C.) | 180 | 86 | 80 |
| 28 | (1.79) | 100% by weight | ketone (5) | | | | |
| Example | 2-methoxy- | activated clay | Methyl ethyl | (45° C.) | 240 | 24 | 58 |
| 29 | (1.79) | 100% by weight | ketone (5) | | | | |
| Example | 2-methoxy- | activated clay | Methyl ethyl | Reflux | 30 | 100 | 98 |
| 30 | (1.79) | 300% by weight | ketone (5) | (75-77° C.) | | | |
| Example | 2-methoxy- | activated clay | Acetonitrile (5) | 78-80° C. | 240 | 59 | 47 |
| 31 | (1.70) | 100% by weight | | | | | |

An amount of a solid acid used (% by weight) is an amount based on the amount of the starting 2-alkoxy-5,9-cyclododecadienone oxime used.
CNCHO^{″}: 12-oxo-4,8-dodecadienenitrile
Activated clay: F-24 available from Engelhard

### Example 32

In 10 ml of methyl ethyl ketone was dissolved 0.98 g (4.39 mmol) of 2-methoxy-5,9-cyclododecadienone oxime, and 0.51 g of activated clay (F-24 available from Engelhard Corporation) which had been dried at 40° C. for 60 minutes under reduced pressure was added to the mixture, and the resulting mixture was reacted under reflux (75 to 77° C.) for 90 minutes. The reaction mixture was filtered, and the activated clay was recovered. When the filtrate was quantitated by GC, a conversion rate of 2-methoxy-5,9-cyclododecadienone oxime was 97 mol %, and 12-oxo-4,8-dodecadienenitrile was found to be formed with a selectivity of 89 mol %.

### Example 33

### First Time Reuse

In 9 ml of methyl ethyl ketone was dissolved 0.89 g (3.99 mmol) of 2-methoxy-5,9-cyclododecadienone oxime, and 0.45 g of activated clay recovered in Example 32 which had been dried at 40° C. for 60 minutes under reduced pressure was added to the mixture, and the resulting mixture was reacted under reflux (75 to 77° C.) for 90 minutes. The reaction mixture was filtered, and the activated clay was recovered. When the filtrate was quantitated by GC, a conversion rate of 2-methoxy-5,9-cyclododecadienone oxime was 97 mol %, and 12-oxo-4,8-dodecadienenitrile was found to be formed with a selectivity of 87 mol %.

### Example 34

### Second Time Reuse

In 8 ml of methyl ethyl ketone was dissolved 0.79 g (3.51 mmol) of 2-methoxy-5,9-cyclododecadienone oxime, and 0.41 g of activated clay (F-24 available from Engelhard Corporation) recovered in Example 33 which had been dried at 40° C. for 60 minutes under reduced pressure was added to the mixture, and the resulting mixture was reacted under reflux (75 to 77° C.) for 90 minutes. When the reaction mixture was quantitated by GC, a conversion rate of 2-methoxy-5,9-cyclododecadienone oxime was 92 mol %, and 12-oxo-4,8-dodecadienenitrile was found to be formed with a selectivity of 89 mol %.

It can be understood that the solid acid(s) of the present invention is also effective in lifetime of the catalyst.

### Example 35

In 10 ml of methyl isobutyl ketone was dissolved 0.40 g (1.79 mmol) of 2-methoxy-5,9-cyclododecadienone oxime, and 0.40 g of H-β type zeolite (Si/Al: 12.5) was added to the mixture, and the resulting mixture was reacted under reflux (105 to 107° C.) for 120 minutes. When the reaction mixture was quantitated by GC, a conversion rate of 2-methoxy-5,9-cyclododecadienone oxime was 94 mol %, and 12-oxo-4,8-dodecadienenitrile was found to be formed with a selectivity of 70 mol %.

### Example 36

In 5 ml of methyl ethyl ketone was dissolved 0.41 g (1.84 mmol) of 2-methoxy-5,9-cyclododecadienone oxime, and 0.41 g of H-USY zeolite (Si/Al: 6) and 0.41 g of diphenyl ether which is an internal standard substance were added to the mixture, and the resulting mixture was reacted under reflux (75 to 77° C.) for 60 minutes. When the reaction mixture was quantitated by GC, a conversion rate of 2-methoxy-5,9-cyclododecadienone oxime was 97 mol %, and 12-oxo-4,8-dodecadienenitrile was found to be formed with a selectivity of 88 mol %.

### Example 37

In 10 ml of methyl ethyl ketone was dissolved 0.39 g (1.47 mmol) of 2-butoxy-5,9-cyclododecadienone oxime, and 0.40 g of activated clay (F-24 available from Engelhard Corporation) was added to the mixture, and the resulting mixture was reacted under reflux (75 to 77° C.) for 120 minutes. When the reaction mixture was quantitated by GC, 2-butoxy-5,9-cyclododecadienone oxime was completely reaction, and 12-oxo-4,8-dodecadienenitrile was found to be formed with a selectivity of 85 mol %.

The results of Examples 32 to 37 are also shown in Table 3 together.

TABLE 3

| | Starting material | | | | | | |
|---|---|---|---|---|---|---|---|
| | 2-Alkoxy-5,9- | | | | | Oxime | |
| | cyclododeca- | | | Reaction | | conversion | CNCHO^{″} |
| | dienone oxime | Solid acid | Solvent | temperature | Reaction | rate | selectivity |
| | (mmol) | (% by weight) | (ml) | (° C.) | time (min) | (mol %) | (%) |
| Example | 2-methoxy- | Activated clay | Methyl ethyl | Reflux | 90 | 97 | 89 |
| 32 | (4.39) | 52% by weight | ketone(10) | (75-77° C.) | | | |
| Example | 2-methoxy- | Activated clay | Methyl ethyl | Reflux | 90 | 97 | 87 |
| 33 | (3.99) | 51% by weight | ketone (9) | (75-77° C.) | | | |
| Example | 2-methoxy- | Activated clay | Methyl ethyl | Reflux | 90 | 92 | 89 |
| 34 | (3.54) | 52% by weight | ketone (8) | (75-77° C.) | | | |
| Example | 2-methoxy- | H-β Si/Al: 12.5 | Methyl isobutyl | Reflux | 120 | 94 | 70 |
| 35 | (1.79) | 100% by weight | ketone (10) | (105-107° C.) | | | |
| Example | 2-methoxy- | H-USY Si/Al: 6 | Methyl ethyl | Reflux | 60 | 97 | 88 |
| 36 | (1.84) | 100% by weight | ketone (5) | (75-77° C.) | | | |
| Example | 2-butoxy- | Activated clay | Methyl ethyl | Reflux | 120 | 100 | 85 |
| 37 | (0.47) | 104% by weight | ketone (5) | (75-77° C.) | | | |

An amount of a solid acid used (% by weight) is an amount based on the amount of the starting 2-alkoxy-5,9-cyclododecadienone oxime used.
CNCHO^{″}: 12-oxo-4,8-dodecadienenitrile
Activated clay: F-24 available from Engelhard

### UTILIZABILITY IN INDUSTRY

According to the present invention, an ω-cyanoaldehyde compound can be prepared by reacting a 2-alkoxy-cycloalkanone oxime compound to bring into contact with a solid acid(s), with safety, simple and easy operations, and high selectivity, and yet, a process which is simple and easy in separating operation of the catalyst after the reaction can be provided.

The obtainable ω-cyanoaldehyde compound is a useful compound as a starting material for various diamines, aminonitriles, etc. For example, 12-oxo-4,8-dodecadienenitrile can be led to dodecamethylene diamine which is useful as a starting material of 1212 Nylon, etc. by reductive amination.

1. A process for preparing an ω-cyanoaldehyde compound which comprises contacting a 2-alkoxycycloalkanone oxime compound and a solid acid(s).

2. The process for preparing an ω-cyanoaldehyde compound according to claim 1, wherein the 2-alkoxycycloalkanone oxime compound is a 2-alkoxycycloalkanone oxime compound a saturated or unsaturated cyclic hydrocarbon having a carbon number of 6 to 12.

3. The process for preparing an ω-cyanoaldehyde compound according to claim 1, wherein the 2-alkoxycycloalkanone oxime compound is one selected from the group consisting of 2-methoxycyclopentanone oxime, 2-methoxycyclohexanone oxime, 2-methoxycyclohexenone oxime, 2-methoxycycloheptanone oxime, 2-methoxycyclooctanone oxime, 2-methoxycyclooctenone oxime, 2-methoxycyclononanone oxime, 2-methoxycyclodecanone oxime, 2-methoxycycloundecanone oxime, 2-methoxycyclododecanone oxime, 2-methoxycyclododecadienone oxime and 2-butoxycyclododecadienone oxime.

4. The process for preparing an ω-cyanoaldehyde compound according to claim 1, wherein the 2-alkoxycycloalkanone oxime compound is a 2-alkoxycyclododecanone oxime compound.

5. The process for preparing an ω-cyanoaldehyde compound according to claim 4, wherein the 2-alkoxycyclododecanone oxime compound is a 2-alkoxy-5,9-cyclododecadienone oxime compound.

6. The process for preparing an ω-cyanoaldehyde compound according to claim 4, wherein the 2-alkoxycyclododecanone oxime compound is 2-methoxy-5,9-cyclododecadienone oxime compound or 2-butoxy-5,9-cyclododecadienone oxime compound.

7. The process for preparing an ω-cyanoaldehyde compound according to claim 1, wherein the solid acid(s) is one which shows characteristics of a Brønsted acid or Lewis acid while it is a solid state.

8. The process for preparing an ω-cyanoaldehyde compound according to claim 1, wherein the solid acid(s) is at least one selected from the group consisting of zeolites and a modified product thereof, oxides, composite oxides, clay mineral, ion exchange resin and a molded product in which these materials are carried on silica gel, phosphoates, sulfates and heteropoly acid.

9. The process for preparing an ω-cyanoaldehyde compound according to claim 1, wherein the solid acid(s) is at least one selected from the group consisting of β type zeolite, mordenite, titanosilicate, MCM-22 and a modified product thereof, aluminum oxide, zinc oxide, SiO₂—Al₂O₃, SiO₂—TiO₂, kaolin, bentonite, activated clay, Amberlyst®, Nafion® and a molded product in which these materials are carried on silica gel, calcium phosphate, sulfated zirconia, copper sulfate and heteropoly acid.

10. The process for preparing an ω-cyanoaldehyde compound according to claim 1, wherein the solid acid(s) is at least one selected from the group consisting of H-β zeolite, activated clay and Nafion® SAC-13.
